(19) United States
(12) Patent Application Publication
(10) Pub. No.: US 2009/0204047 A1
(43) Pub. Date: Aug. 13, 2009

(54) Quick Release Casts

(75) Inventor: Robert MacArthur, Irvine, CA (US)

Correspondence Address: Robert MacArthur, 85 W. La Veta, #104, Orange, CA, 92868, US

(73) Assignee: MacArthur

(21) Appl. No.: 12/246,285

(22) Filed: Oct. 6, 2008

Related U.S. Application Data
(60) Provisional application No. 61/065,113, filed on Feb. 8, 2008.

(51) Int. Cl. A61F 5/00

(52) U.S. Cl. 602/36; 602/5; 602/6

(57) ABSTRACT

Quick-release casts are presented. A cast can include multiple layers of material. An inner layer comprising a soft material contacts an affected limb and forms a padded layer. One or more additional layers can wrap around the padded layer to form nested tubular casts. The materials used to form the tubular casts are preferably impregnated with a curable compound capable of hardening to provide rigid support for the limb. Each of the tubular casts can wrap around the affect limb and have an overlapping seam that permanently seals in position upon curing of the curable compound. Each of the tubular casts can include a quick-release fastener that pre-joins sections of the tubular casts together before application to the limb. After the limb heals, the fastener can be undone to remove the cast quickly, quietly, and comfortably without causing undue distress to the patient.

This application claims the benefit of priority to U.S. provisional application having Ser. No. 61/065,113 filed on Feb. 8, 2008. This and all other extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply.

### FIELD OF THE INVENTION

The field of the invention is casting technologies.

### BACKGROUND

Casts are applied to a broken or a weakened limb of patient to promote proper healing. Typically, casts comprise a material that can be hardened (e.g., polymer or plaster) to form a rigid support structure for the limb. A medial practitioner uses a cast cutting saw to remove the cast by cutting through layers of the cast. Such cutting tools can cause unnecessary trauma to young patients or animals. The cutting tool is simply too loud, generates an uncomfortable amount of heat due to friction, or takes too long to cut off the cast.

For example, U.S. Pat. No. 6,585,671 to Rhee titled “Hybrid-Mesh Cast Sleeve and Method” filed on Sep. 11, 2001, describes a cast sleeve impregnated with a hardening agent. Once the sleeve is deployed around a body part of a person or an animal, the hardening agent can be solidified to form a tubular cast. The solid tube cast must be removed using a cutting tool. Although the Rhee approach has use in some areas, Rhee fails to address the need for a cast that can be removed quickly, quietly, and comfortably.

Some progress has been made to create casts that are more comfortable to remove. For example, U.S. Pat. No. 5,415,622 to Kelly titled “Easily Removed Tubular Cast Assembly and Method for Removing a Cast” filed on Dec. 21, 1993, utilizes a single piece tubular sleeve that has a weakness zone. The sleeve can be placed over an affected body part and then harden to form a cast. The sleeve has a pre-formed weakness zone along the length of the cast. To remove the cast, a practitioner uses scissors or a saw to cut along the weakness zone. Although the Kelly approach offers a slightly easier approach to removing a cast, the weakness zone could allow the cast to fracture prematurely. Additionally the tools required to remove the cast could still cause a young patient or an animal distress.

Still others have attempted to produce casts that can be easily removed without the use of tools. Examples include U.S. Pat. No. 6,093,161 to Vlaeyen et al. titled “Thermoplastic Apparatus with Fastener” filed on Dec. 6, 1995, and U.S. Patent Publication 2004/0147859 to O'Keefe titled “Orthopedic Cast Construction” filed on filed on Feb. 25, 2002. In both cases, the casts have seams that are joined by fasteners. Unfortunately, the seams are weak points that could become undone prematurely or could be easily removed by the patient.

U.S. Pat. No. 4,727,865 to Hill-Byrne titled “Replaceable Rigid Cast with Integral Fasteners” filed on Mar. 7, 1985, offers a stronger fastener system that joins parts of a rigid cast. The fasteners are adjustable buckles that lock into position. The Hill-Byrne approach offers a strong seam; however, the buckles protrude from the surface of the cast and can become snagged or caught on clothing or other objects.

The above cast systems also fail to address the need for a cast that can be fitted to a patient's limb in a manner that is neither too tight nor too loose. An ideal cast would allow a practitioner to fit a cast to patient properly without having to undue various fasteners. The ideal cast would also allow a practitioner to remove the casts quickly, quietly, and comfortably.

It has yet to be appreciated that a cast can be constructed having a quick-release fastener that joins sections of the cast at a location other than a seam where the casts seals against itself. Such a cast allows a practitioner to apply the cast and to adjust the position of the seam for a proper fit. The cast can then be hardened to permanently seal the seam. When healing is complete, the practitioner can simply release the quick-release fastener to remove the cast without causing undue stress to the patient.

Thus, there is still a need for quick-release cast that can be removed quickly, quietly, and comfortably by hand.

### SUMMARY OF THE INVENTION

The present invention provides apparatus, systems and methods in which a quick-release cast can be applied to a limb and can be quickly removed without discomfort to a patient. In one aspect of the inventive subject matter, a quick-release cast comprises an outer water impermeable layer having two or more sections of a material impregnated with a curable compound. At least two of the sections are joined together before application of the cast to the limb via a quick-release fastener. The quick-release cast also preferably comprises an inner water permeable layer made of a soft material. The inner permeable layer wraps around the affected limb to form a padded layer. The outer impermeable layer wraps around the padded layer to form an outer tube cast where the sections of the outer impermeable layer have an overlapping region. When the curable compound is cured, the outer tube cast hardens and the sections permanently seal along the overlapping region.

In some embodiments a quick-release cast also comprises an intermediary layer disposed between the padded layer and the outer tube cast. The intermediary layer preferably comprises an inner water impermeable layer of a material impregnated with a curable compound. The inner impermeable layer wraps around the padded layer to form an inner tube cast. The soft material of the padded layer can fold over the open ends of the inner tube cast to form a padded cuff. The outer impermeable layer of material can wrap around the inner tube cast and the folded cuffs to form the outer tube cast. The inner tube cast can also include a quick-release fasten to further enable a quick, quiet, and comfortable removal of the cast.

The quick-release fasteners used within the cast system are preferably mechanic fasteners that can be released quietly (e.g., less than 50 decibels), quickly (e.g., in less than 10 seconds), and comfortably (e.g., less than a 5 degree temperature change). Example quick-release fasteners include zippers, rod-and-eyelet assemblies, rip-cords, or other fasteners. In some embodiments, the fasteners can comprises a one-time-use fastener where once the fastener is removed, it can not be used to rejoin the cast. In especially preferred embodiments, the quick-release fasteners are camouflaged or hidden from view in a manner where an outer tube cast appears to be a substantially continuous section of material.

Various objects, features, aspects and advantages of the inventive subject matter will become more apparent from the following detailed description of preferred embodiments, along with the accompanying drawings in which like numerals represent like components.

### BRIEF DESCRIPTION OF THE DRAWING

FIG. 1 is a schematic of a basic exemplary cast system before application to a limb.

FIG. 2 is a schematic of a cast system configured to use on a human's forearm.

FIG. 3A is a schematic of an initial step during application of a contemplated cast.

FIG. 3B is a schematic of an intermediary step during application of the cast of FIG. 3A.

FIG. 3C is a schematic of the cast of FIG. 3A after application.

FIG. 4. is a schematic of a cast applied to a forearm showing quick-release fastener.

### DETAILED DESCRIPTION

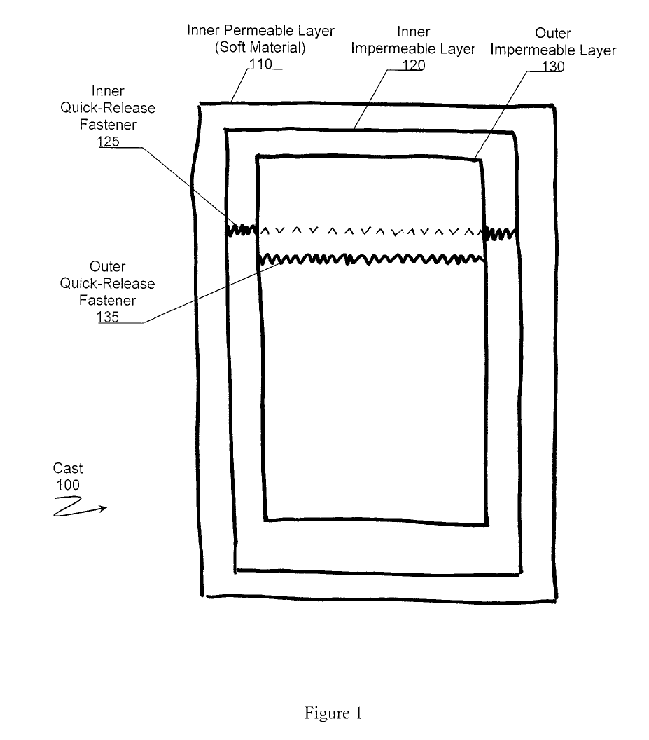

In FIG. 1, cast 100 is shown in a laid flat open state before a practitioner applies cast 100 to a limb. Cast 100 comprises a plurality of layers including inner water permeable layer 110, outer water impermeable layer 130, or inner water impermeable layer 120. Preferably one or more quick-release fasteners are used to pre-join sections of the layers before the cast is applied. For example, inner impermeable layer 120 comprises inner quick-release fastener 125 and outer impermeable layer 130 comprises outer quick-release fastener 135. The inner impermeable layer 120 and outer impermeable layer 130 preferably are impregnated with a curable compound that hardens upon curing. Each layer can be sequentially wrapped around an affected to form tubular cast structures. Once in place, the compound can be cured to harden the cast. It should be noted that the number of layers, their arrangement relative to each other, or their sizes and dimensions can be adjusted as desired while still falling within the scope of the inventive subject matter.

In a preferred embodiment, inner permeable layer 110 comprises a soft, flexible material that can be comfortably placed against an affected limb. When cast 100 is applied to the limb, the soft material preferable wraps around and is in contact with the limb to form a padded interior layer of cast 100. The padded layer preferably allows the limb to “breath”. Suitable soft material includes cotton cast padding, acrylic cast padding, a portion of stockinette, or other soft materials known or yet to be invented. In a preferred embodiment, the soft materials are permeable to water yet impermeable to a curable compound impregnating an adjacent outer layer. One should note that layer 110 can comprise a plurality of flexible materials as desired to form a padded layer, as opposed using a single material.

Inner permeable layer 110 can also include one or more fasteners that can be used to securely fasten the soft material to a limb. Example fasteners include mechanical fasteners (e.g., snaps, zippers, hook-and-loop fasteners, etc. . . . ), chemical fasteners (e.g., adhesive, glue, etc. . . . ), or other types of fasteners. In embodiments where layer 110 includes fasteners, the fasters are preferably flexible and thin to reduce the discomfort of the wearer.

Inner permeable layer 110 can couple to outer impermeable layer 130 directly or indirectly. For example, layer 110 can couple to layer 130 via glue, stitching, or other direct coupling methods. In a more preferred embodiment, layer 110 indirectly couples to layer 130 via an intermediary layer, possibly inner permeable layer 120, as described below.

Outer impermeable layer 130 preferably comprises a flexible material impregnated with a curable compound. Acceptable flexible material that can be used to form outer impermeable layer 130 can include cotton padding, fiberglass, cloth, or other flexible materials. Acceptable curable compounds can include those that are cured through chemical reaction, exposure to light, thermal exposure, or through exposure to other energy sources. In a preferred embodiment, the curable compound can be cured to a desired hardness in a manner that is comfortable to the patient. For example, the compound can comprise a polymer or plaster that is cured through exposure to body heat, exposure to ultraviolet light sources, or simply dries when exposed to air. Such materials can be cured without subjecting the patient to undue mess or temperature changes.

Materials for forming an outer layer 130 are well known. Example materials that are suitable for use or can be adapted for use in producing outer layer 130 are described in the following literature:
U.S. Pat. No. 5,027,803 to Scholz et al. titled “Orthopedic Splinting and Casting Article” filed on Jul. 22, 1988.
U.S. Pat. No. 5,273,802 to Scholz et al. titled “Orthopedic Casting Materials Having Superior Lamination Characteristics Due to Napped Surface” filed on Jul. 7, 1989.
U.S. Pat. No. 5,931,798 to Green et al. titled “Orthopedic Casting Tape Having Alternating Thick and Thin Portions” filed on May 7, 1993.
U.S. Pat. No. 6,159,877 to Scholz et al. titled “Fabric Backing for Orthopedic Support Materials” filed on Feb. 13, 1998.

In a preferred embodiment, outer impermeable layer 130 includes quick-release fastener 135 that pre-joins two sections of layer 130 before cast 100 is applied to a limb. The two sections of layer 130 are pre-joined via fastener 135, preferably at manufacturing time. Having the sections of layer 130 pre-joined allows a practitioner to wrap layer 130 around the padded layer and the affected limb as desired to ensure a proper fit, as opposed to having to adjust a fastener along a seam as required in previous cast systems.

Quick-release fastener 135 preferably runs from one edge of outer impermeable layer 130 to an opposing edge. When cast 100 is to be removed, a practitioner can simply undue fastener 135 to open cast 100 and remove cast 100 from the limb. Fastener 135 can include mechanical, chemical, or other types of fasteners. Preferred fasteners are mechanical fasteners that can be quietly, quickly, and comfortably undone. For example, fastener 135 can comprise a series of eyelets on adjacent edges of the sections of layer 130 where a flexible rod runs through the eyelets to join the two sections. The rod-eyelet assembly can also function as a hinge while applying cast 100 to a limb. Upon removal cast 100, the rod can be pulled from the eyelets to allow cast 100 to open. The rod-eyelet assembly allows the cast to be removed quietly (e.g., generates a sound of less than 50 decibels) without causing undue distress to the patient as opposed to a traditional cast requiring a loud saw. Additionally, the rod-eyelet assembly allows the cast to be removed comfortably without substantially altering a temperature (e.g., less than a five degree Celsius change) of cast 100 or limb as would ordinarily occur due to friction of a saw. Furthermore, the rod-eyelet assembly can be undone nearly instantaneously by simply pulling the rod out, which separates the adjacent sections and allows cast 100 to be removed quickly (e.g., in less than 10 seconds) by sliding cast 100 of the affected limb.

Fastener 135 can take on other acceptable forms beyond a rod-eyelet assembly. Other examples of fasteners that can be removed quickly, quickly, and comfortably include a hook-and-loop fasteners, zippers, snaps, or a rip-cord. In some embodiments, fastener 135 is a one-time-use fastener where once the fastener is undone, the fastener can not be fastened. An example of a one-time-use fastener includes a rip-cord fastener that stitches the seconds of layer 130 together. Once the rip-cord is pulled, the layer of the cast is split up and can be quickly, quietly, and comfortably removed.

It should be appreciated that outer impermeable layer 130 can comprise more than one of fastener 135. In embodiment having multiple fasteners on an outer layer further enhances the removability of cast 100. For example, a first fastener 135 and a second fastener 135 can be positioned on outer layer 130 where the two fasteners are on opposite sides of a limb. Cast 100 can be quickly removed by splitting cast 100 into multiple parts by undoing both fasteners as opposed to removing cast 100 by bending or breaking cast 100 as might be required when a single fastener 135 is deployed.

In a preferred embodiment, cast 100 also includes an intermediary layer disposed between inner permeable layer 110 and out impermeable layer 130. For example, the intermediary layer can include inner impermeable layer 120. Inner impermeable layer 120 offers one or more advantages for cast 100 including adding additional rigid support for a limb, or simply holding inner permeable layer 110 in place.

Inner impermeable layer 120 can preferably comprise similar, possibly the same, materials as outer impermeable layer 130. However, it is also contemplated that different materials or different curable compounds, if any, could be used for layer 120 depending on the target application. In a preferred embodiment, layer 120 includes the same material and curable compounds used to produce layer 130.

It is also contemplated that inner impermeable layer 120 includes an inner quick-release fastener 125. However, inner quick-release fastener 125 is likely obscured from view by a patient once outer permeable layer 130 wraps around layer 120 when cast 100 is applied to a limb. Therefore, fastener 125 could be different from fastener 135. For example, fastener 135 might comprise a rip-cord embedded in the materials of layer 130 where the rip-cord is hidden from view. Fastener 125 might simply comprise a hook-and-loop fastener (e.g., VELCRO™ brand fastener). As in layer 130, layer 120 could also comprise more than on fastener 125.

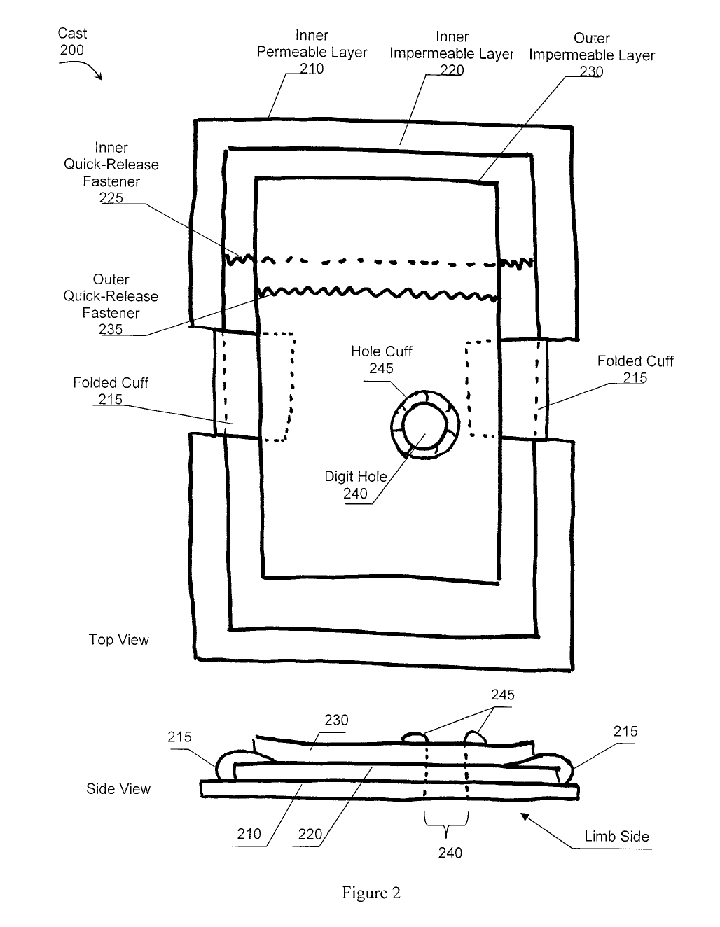

In FIG. 2, cast 200 presents an exemplary contemplated inventive cast configured for use on a forearm of a human. FIG. 2 shows cast 200 laid open before being applied to a limb and illustrates cast 200 via a top view and a side view.

Cast 200 comprises inner permeable layer 210 to be placed against the forearm, inner impermeable layer 220 to form an inner tube cast, and outer impermeable layer 230 to form an outer tube cast. In the example shown, both layer 220 and layer 230 include fasteners 225 and 235, respectively. Layers 210, 220, and 230 can couple to each other a priori before application, if desired, by using an adhesive, stitching, or simply through folded material as shown with cuffs 215 and 245.

Cast 200 also preferably includes digit hole 240 for receiving a digit of a hand, a thumb for example. Hole 240 can be formed by aligning co-extensive holes in layers 210, 220, and 230. In some embodiments the soft material composing inner permeable layer 210 can extend through hole 240 to form hole cuff 245, which can also couple layer 210 to layer 230. Cuff 245 provides a soft, padded edge to protect a patient from rough edges of hole 240.

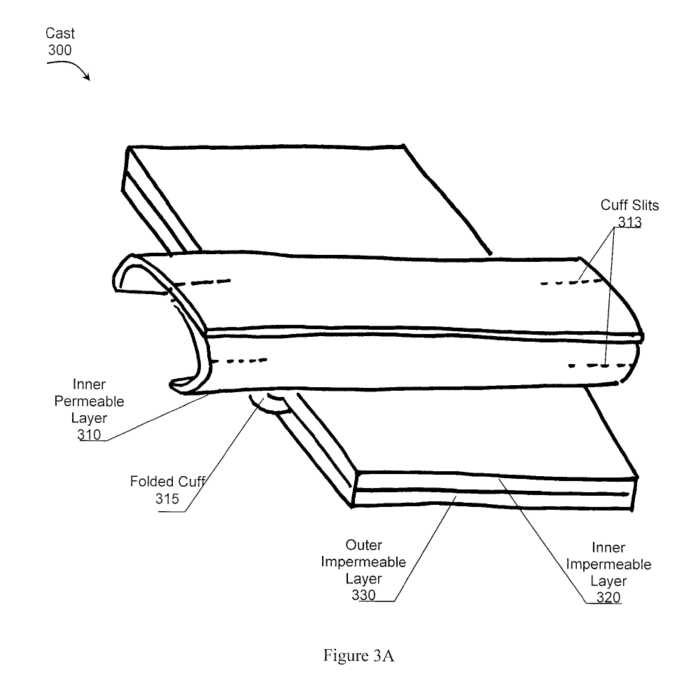

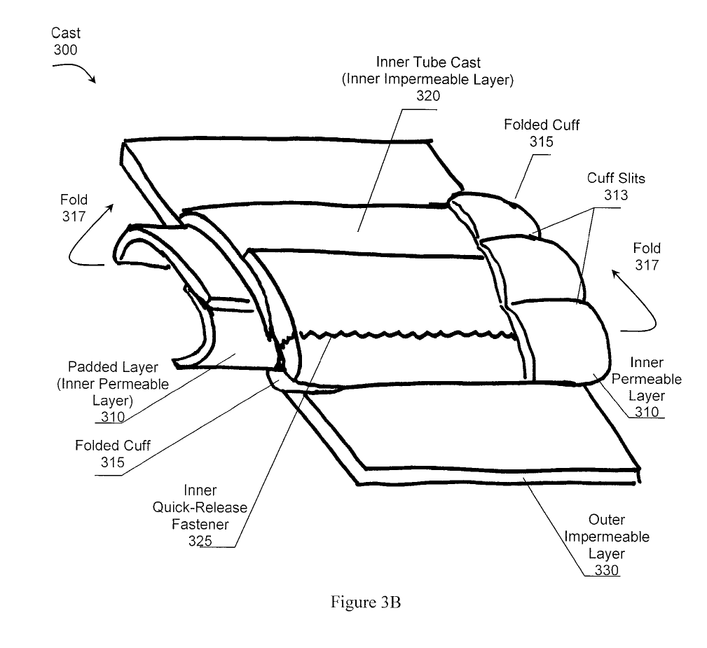

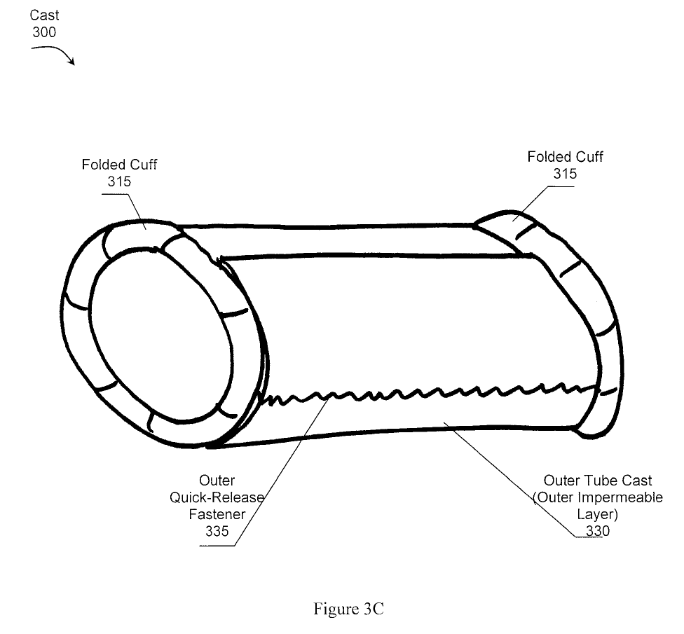

In some embodiments, the soft material used to form inner permeable layer 210 can also include cut sections that fold over inner impermeable layer 220 and tucks under outer impermeable layer 230. The pre-folded soft material forms folded cuffs 215 that protect the patient from rough edges that become rigid when the curable compound in layers 220 and 230 hardens. As cast 200 is applied to an affected limb, excess soft material extending beyond the edges of layer 220 can also be folded over as discussed below with respect to FIG. 3B.

Although cast 200 is depicted for use on a forearm of a human being, it should be noted that cast 200 can be configured to fit any limb including arms or legs. Furthermore, cast 200 can be configured for fit appendages or limbs of non-human animals, possibly a dog, a cat, a cow, a horse, or other animals considered to have limbs.

One should also note that cast 200 can be produced in different sizes or shapes to accommodate limbs of different sizes or shapes. For example, casts can be manufactured in different sizes to accommodate infants, children, or adults. It is also specifically contemplated that cast 200 can be produced where layers 210, 220, or 230 are pre-contoured to fit a limb. For example cast 200 could include contours that correspond to a curve of a wrist, a hand, a elbow, ankle, foot, or other body curve.

FIGS. 3A, 3B, and 3C illustrate various stages of applying an exemplary cast 300 to a limb. The limb is not shown for clarity.

FIG. 3A illustrates an initial step of applying cast 300. The soft inner permeable layer 310 wraps around an affected limb to form a tubular padded layer around the limb and that is preferably in contact with the limb. A practitioner can adjust to the layer 310 to a desired snug fit. In some embodiments, layer 310 can include a fastener to hold the material of layer 310 in place. For example, layer 310 can include hook-and-loop fasteners or could simply be cohesive.

The soft material comprising the layer 310 can include a plurality of cuff slits 313, which allow the soft material to fold easily. The soft material preferably includes pre-folded cuffs 315 on opposing sides of cast 300 as previously discussed.

FIG. 3B illustrates an intermediary step of applying cast 300 where inner impermeable layer 320 preferably wraps around the limb and the padded tube layer created by layer 310. Layer 320 forms an inner tube cast of cast 300. Once layer 320 wraps around the limb, layer 320 preferably comprises a region where portions of layer 320 overlap. When the curable compound of layer 320 cures or hardens, the overlapping portion of layer 320 permanently seals in position. In a preferred embodiment, the sealed seam is approximately as strong as the reaming cured portions of layer 320. As used herein, “permanently seals” should be interpreted to mean coupling a seam or other overlapping region in a manner where the seam can not be undone without breaking the seam.

In some embodiments, the inner tube cast formed by layer 320 comprises inner quick-release fastener 325. When healing is completed, fastener 325 can be released to remove inner tube cast quickly, quietly, and comfortably.

Preferably, excess soft material composing layer 310 extending beyond the ends of the inner tube cast is folded by folds 317 to form folded cuffs 315. The material can be folded easily over ends of inner tube cast easily due to pre-cut cuff slits 313.

FIG. 3C shows a final stage of applying cast 300. Outer layer 330 wraps around the inner layers (e.g., layers 310 or layer 320), to form an outer tube cast. Layer 330 also holds the soft material forming cuff 315 in position. Cuffs 315 preferably pad rough edges of the inner and outer tube casts to protect the patient from possible irritation or discomfort.

Once layer 330 is wrapped around the limb, layer 330 also preferably comprises an overlapping region where sections of material composing layer 330 overlap. When the material of layer 330 is cured, the overlapping sections become permanently sealed along the overlapping region. The permanent seal along the seam defined by the overlapping region reduces the risk of cast 300 becoming undone as could happen with previous casting systems using fasteners along a joining seam. Furthermore, in a preferred embodiment, outer quick-release fastener 335 is accessible near the outer surface of layer 330 and is located in position other than the permanently sealed overlapping region. It should be noted that fastener 335 is shown as visible. However, it is specifically contemplated that fastener 335 can be hidden or camouflaged from view to reduce a risk of premature or accidental removal of vast 300.

After forming the outer cast tube, cast 300 can be allowed to cure. In embodiments employing plaster, curing can occur naturally without requiring additional actions by a practitioner. In other embodiments using different curable compounds, curing can occur by exposing the curable compound a suitable energy source (e.g., heat, UV light, etc. . . . ).

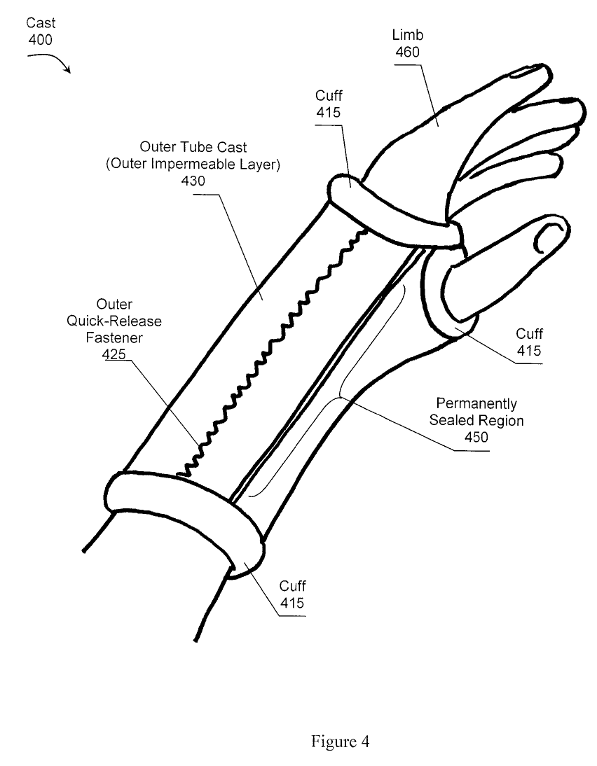

FIG. 4 illustrates a cast 400 applied to a limb 460, a forearm of a human. Outer tube cast 430 is rigid and cuffs 415 protect the patient from the hardened rough edges of cast 400. Preferably, outer tube cast 430 is permanently sealed along region 450 after the curable compound within outer tube cast 430 hardens. Cast 400 can be removed quickly, quietly, and comfortably by simply releasing fastener 425 as previously described.

One should note that fastener 425 and sealed region 450 are not required to be co-extensive. An advantage of placing faster 425 in a different position than the seam formed by region 450 is clear. A practitioner can form outer tube cast around the affected limb 450 to a desired fit that is not too tight or not too loose as could happen with cast systems having fasteners along the seam. Additionally, the cast can be quickly removed by undoing fastener 425 by hand without the use a cutting saw, scissors, or other mechanical tool.

Sealed region 450 is shown within FIG. 4 as a longitudinal seam running the length of and joining sections of the outer tube cast 430. However, sealed region 450 can be of any size or shape. In some embodiments, region 450 can comprise a jagged or irregular seam. In yet other embodiments, the sections of outer tube cast 430 that are permanently sealed by region 450 can include strips of casting material (e.g., casting tape, gauze, bandages, etc. . . . ) as opposed to a single continuous sheet as shown.

In a preferred embodiment, fastener 425 is at least flush with the surface of outer tube cast 430 to prevent cast 400 from accidentally snagging on clothing or catching against objects as can happen in known cast systems.

Although FIG. 4 shows quick-release fastener 425 as being visible, it is specifically contemplated that fastener 425 can be camouflaged or hidden from view. In a preferred embodiment, fastener 425 can be embedded into the material composing outer tube cast 430 in manner where the outer impermeable layer of outer tube cast 430 appears to be a single continuous section that hides the fastener from view.

It is also contemplated that outer tube cast 430 can be coated or covered with a coating layer to hide fastener 425. Preferred coating layers include substances that are receptive to art work. Such an approach allows individuals to sign or to draw on cast 400 easily. Furthermore, a coating layer can include decorative options, possibly targeting children. Coating layers can include licensed characters, optional colors, optional patterns, or other interesting features.

Hiding fastener 425 has several advantages. One advantage is that a continuous uniform surface provides for an aesthetically pleasing cast. Another more relevant advantage includes reducing the risk that the patient will release fastener 425 prematurely.

In a preferred embodiment, cast 400 is prepared for application to an affected limb at manufacturing time and pre-packaged within a container. Preferred containers include those that prevent premature curing of the materials that are impregnated with a curable compound. For example, a container can include an opaque, water tight bag where the bag prevents any curable compounds from drying or possibly being exposed to UV light or other curing energy sources. Additionally, each layer of cast 400 can be separated from adjacent layers by non-reactive sheets of material to prevent the layers from accidentally bonding with each other.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A quick-release cast comprising:
an outer impermeable layer comprising a first and a second section of a first material impregnated with a curable compound where the sections are pre-joined via a first quick-release fastener;
an inner permeable layer comprising a soft material coupled to the outer impermeable layer;
wherein the inner permeable layer wraps around and is in contact with a limb to form a padded layer; and
wherein the outer impermeable layer wraps around the padded layer in a manner where the first section overlaps a region of the second section, and where upon curing of the curable compound the first section and the second section become permanently sealed along the region and the outer impermeable layer forms a hardened outer tube cast.

2. The cast of claim 1, further comprising an inner impermeable layer of the first material impregnated with the curable compound disposed between the inner permeable layer and the outer impermeable layer, and that wraps around the padded layer forming an inner tube cast, and where the soft material folds over the open ends of the inner tube cast to form a padded cuff held in place by the outer tube cast that wraps around the inner tube cast.

3. The cast of claim 2, wherein inner tube cast comprises an overlapping portion, and comprises a second quick-release fastener located in a position other than the overlapping portion.

4. The cast of claim 3, wherein upon curing of the inner impermeable layer the inner tube cast hardens and the overlapping portion permanently seals in position.

5. The cast of claim 3, wherein the second quick-release fastener is different than the first quick-release fastener.

6. The cast of claim 3, wherein the second quick-release fastener is a hook and loop fastener.

7. The cast of claim 1, wherein the inner permeable layer and the outer impermeable both comprise approximately coextensive holes for receiving a digit of the limb.

8. The cast of claim 7, wherein the receiving hole comprises a padded cuff formed of the soft material.

9. The cast of claim 7, wherein the digit is a thumb.

10. The cast of claim 1, wherein the inner permeable layer and the outer impermeable layer are both per-contoured to fit a limb.

11. The cast of claim 1, wherein the limb is selected from the group consisting of an arm and a leg.

12. The cast of claim 1, wherein the limb belongs to a non-human animal.

13. The cast of claim 1, wherein the quick-fastener comprises a one-time-use fastener.

14. The cast of claim 13, wherein the quick-release fastener comprises a rip-cord.

15. The cast of claim 1, wherein the quick-release fastener comprises a zipper.

16. The cast of claim 1, wherein the quick-release fastener comprises a rod and eyelet assembly.

17. The cast of claim 1, wherein the outer tube cast and the padding layer are hand-removable without requiring a tool.

18. The cast of claim 17, wherein a sound made by the first quick-release fastener during removal has a loudness no greater than 50 decibels.

19. The cast of claim 17, wherein a temperature change of the outer tube cast induced by the first quick-release fastener during removal is no greater than five degrees Celsius.

20. The cast of claim 17, wherein the outer tube cast and the padding layer can be removed in less than 10 seconds.

21. The cast of claim 1, wherein the outer impermeable layer appears to be a single continuous section that hides the first quick-release fastener from view.
